(19) United States
(12) Patent Application Publication
(10) Pub. No.: US 2025/0195837 A1
(43) Pub. Date: Jun. 19, 2025

(54) MEDICAL INSTRUMENT WITH COMPONENT MOVEMENT TRACKING AND REAL-TIME POSITION SENSING

(71) Applicant: Acclarent, Inc., Irvine, CA (US)

(72) Inventors: Cesar FUENTES-ORTEGA, Pasadena, CA (US); Shubhayu BASU, Anaheim, CA (US)

(21) Appl. No.: 18/986,452

(22) Filed: Dec. 18, 2024

Related U.S. Application Data
(60) Provisional application No. 63/611,230, filed on Dec. 18, 2023.

(51) Int. Cl. A61M 25/01; A61B 17/24; A61B 34/20; A61M 25/09; A61M 29/02

(52) U.S. Cl. CPC A61M 25/0113; A61M 29/02; A61B 17/24; A61B 2034/2072; A61M 2025/0166; A61M 25/09; A61M 2029/025

(57) ABSTRACT

A dilation instrument can be configured to provide real-time tracking of components of the instrument during a surgical procedure, such as by providing real-time tracking of respective positions of the components during the procedure. The dilation instrument can include a body, a shaft assembly, a guide element, a working element, and a movement tracking feature. The guide element can translate relative to the body and have a position sensor that can provide a signal indicating a position of the guide element in three-dimensional space. The working element can translate relative to the body and the guide element. The movement tracking feature can generate a signal indicating movement of the working element relative to the body and/or the guide element.

### CROSS-REFERENCE TO RELATED APPLICATIONS

This application claims the benefit of U.S. Provisional Application No. 63/611,230, filed Dec. 18, 2023, the entirety of which is incorporated herein by reference.

### BACKGROUND

There are various circumstances in which it may be necessary to dilate an anatomical passageway in a patient. For example, a patient may require the dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guidewire and catheter to position an inflatable balloon within the anatomical passageway, then inflating the balloon with a fluid (e.g., saline) to dilate the anatomical passageway.

For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pat. Nos. 11,534,192, 9,579,448, 9,155,492, and U.S. Pub. No. 2021/0361912, the entirety of each of which is incorporated by reference herein.

### SUMMARY OF THE INVENTIONS

In the context of Eustachian tube dilation, a dilation catheter or other dilation instrument may be inserted into the Eustachian tube and then inflated or otherwise expanded to dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Pat. Nos. 10,206,821 and 11,013,896, the entirety of each of which is incorporated herein by reference.

Some medical instruments may include an adjustable guide that allows the same medical instrument to readily access different anatomical structures (e.g., Eustachian tubes, different passageways associated with drainage of paranasal sinuses, etc.). Examples of dilation instruments with adjustable guides are described in U.S. Pat. Nos. 10,137,285, 11,013,897, and 11,534,192, the entirety of each of which is incorporated herein by reference.

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images.

An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map.

During IGS procedures, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

Nevertheless, in accordance with at least some embodiments disclosed herein is the realization that such IGS systems do not provide adequate or accurate real-time tracking of specific aspects and components of the system. For example, some embodiments disclosed herein include the realization that numerous advantages can be achieved during surgery if such real-time tracking information is provided, such as real-time tracking of the respective positions of various components of an ENT instrument in a patient. Due to significant restrictions in size and complexity, conventional systems have not been able to provide such features and advantages. Thus, systems and components within the scope of the embodiments disclosed herein provide a significant advance and benefit due to the unique configurations and designs that have been created and described in the present disclosure. While several systems and methods have been made and used to track an ENT instrument in a patient, it is believed that no one prior to the inventors has made or used the inventions described in the appended claims.

### BRIEF DESCRIPTION OF THE DRAWINGS

The drawings and detailed description that follow are intended to be merely illustrative and are not intended to limit the scope of the inventions as contemplated by the inventors.

FIG. 1 depicts a schematic view of an example of a surgery navigation system being used on a patient seated in an example of a medical procedure chair, in accordance with some embodiments.

FIG. 2 depicts a perspective view of an example of a medical instrument, with a dilation catheter in a proximal position, in accordance with some embodiments.

FIG. 3 depicts an exploded perspective view of components of a shaft assembly of the instrument of FIG. 2, in accordance with some embodiments.

FIG. 4 depicts a schematic view of a distal portion of a guide element of the shaft assembly of FIG. 3, in accordance with some embodiments.

FIG. 5 depicts a perspective view of an actuator of the instrument of FIG. 2, in accordance with some embodiments.

FIG. 6 depicts a perspective view of an actuation assembly of the instrument of FIG. 2, in accordance with some embodiments.

FIG. 7A depicts a cross-sectional side view of a portion of the instrument of FIG. 2, in an example of a first state of operation, in accordance with some embodiments.

FIG. 7B depicts a cross-sectional side view of a portion of the instrument of FIG. 2, in an example of a second state of operation, in accordance with some embodiments.

FIG. 7C depicts a cross-sectional side view of a portion of the instrument of FIG. 2, in an example of a third state of operation, in accordance with some embodiments.

FIG. 7D depicts a cross-sectional side view of a portion of the instrument of FIG. 2, in an example of a fourth state of operation, in accordance with some embodiments.

FIG. 7E depicts a cross-sectional side view of a portion of the instrument of FIG. 2, in an example of a fifth state of operation, in accordance with some embodiments.

FIG. 8A depicts a distal portion of the shaft assembly of FIG. 3, while the instrument of FIG. 2 is in the first state of operation of FIG. 7A, in accordance with some embodiments.

FIG. 8B depicts a distal portion of the shaft assembly of FIG. 3, while the instrument of FIG. 2 is in the second state of operation of FIG. 7B, in accordance with some embodiments.

FIG. 8C depicts a distal portion of the shaft assembly of FIG. 3, while the instrument of FIG. 2 is in the third state of operation of FIG. 7C, in accordance with some embodiments.

FIG. 8D depicts a distal portion of the shaft assembly of FIG. 3, while the instrument of FIG. 2 is in the fourth state of operation of FIG. 7D, in accordance with some embodiments.

FIG. 8E depicts a distal portion of the shaft assembly of FIG. 3, while the instrument of FIG. 2 is in the fifth state of operation of FIG. 7E, in accordance with some embodiments.

FIG. 9 depicts a partially exploded view of the instrument of FIG. 2, with the actuator removed from the handle, and with capacitor plate features added to the actuator and the handle, in accordance with some embodiments.

FIG. 10A depicts a perspective view of the actuator and handle of FIG. 9, with the actuator in a proximal position along the handle, in accordance with some embodiments.

FIG. 10B depicts a perspective view of the actuator and handle of FIG. 9, with the actuator in a distal position along the handle, in accordance with some embodiments.

FIG. 11A depicts a schematic view of portions of the shaft assembly of FIG. 3, with capacitor plate features added to a dilation catheter shaft assembly and guide rail of the shaft assembly, and with the dilation catheter shaft assembly in a proximal position along the guide rail, in accordance with some embodiments.

FIG. 11B depicts a schematic view of the dilation catheter shaft assembly and guide rail of FIG. 11A, with the dilation catheter shaft assembly in a distal position along the guide rail, in accordance with some embodiments.

FIG. 12A depicts a perspective view of another example of a medical instrument, with a guidewire in a proximal position and a dilation catheter in a proximal position, in accordance with some embodiments.

FIG. 12B depicts a perspective view of the medical instrument of FIG. 12A, with the guidewire in a distal position and the dilation catheter in the proximal position, in accordance with some embodiments.

FIG. 12C depicts a perspective view of the medical instrument of FIG. 12A, with the guidewire in the distal position and the dilation catheter in a distal position, and with a balloon of the dilation catheter in a non-inflated state, in accordance with some embodiments.

FIG. 12D depicts a perspective view of the medical instrument of FIG. 12A, with the guidewire in the distal position and the dilation catheter in the distal position, and with a balloon of the dilation catheter in an inflated state, in accordance with some embodiments.

FIG. 13A depicts a schematic view of the guidewire and dilation catheter if FIG. 12A, with the guidewire in the proximal position and the dilation catheter in the proximal position, in accordance with some embodiments.

FIG. 13B depicts a schematic view of the guidewire and dilation catheter if FIG. 12A, with the guidewire in the distal position and the dilation catheter in the proximal position, in accordance with some embodiments.

FIG. 13C depicts a schematic view of the guidewire and dilation catheter if FIG. 12A, with the guidewire in the distal position and the dilation catheter in the distal position.

### DETAILED DESCRIPTION

The following description of certain examples of the inventions should not be used to limit the scope of the present inventions. Other examples, features, aspects, embodiments, and advantages of the inventions will become apparent to those skilled in the art from the following description, which is by way of illustration, one of the best modes contemplated for carrying out the inventions. As will be realized, the inventions are capable of other different and obvious aspects, all without departing from the inventions. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms, such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments, such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

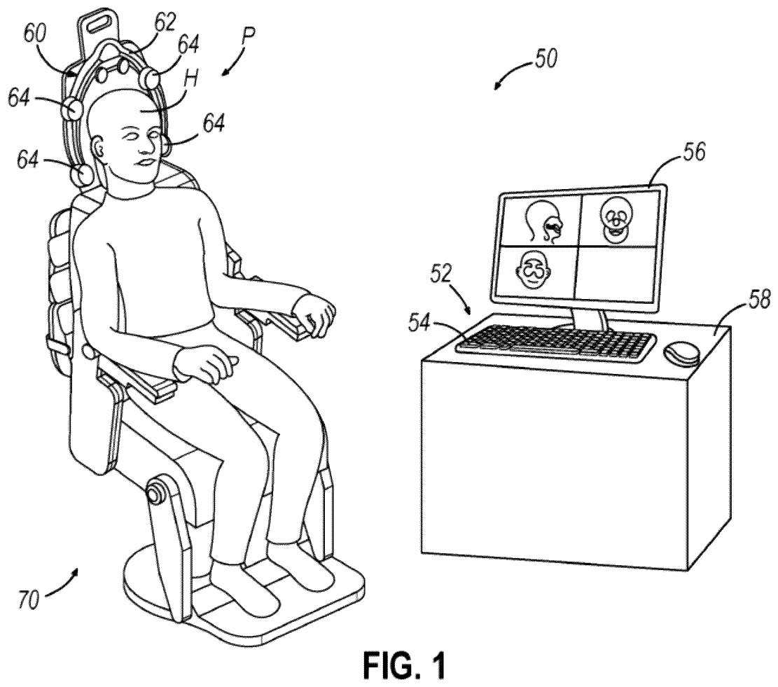

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system 50 enabling a medical procedure to be performed within a head (H) of a patient (P) using image guidance. In addition to or in lieu of having the components and operability described herein, the IGS navigation system 50 may be constructed and operable in accordance with at least some of the teachings of U.S. Pat. Nos. 7,720,521 and 11,065,061, the entirety of each of which is incorporated herein by reference.

The IGS navigation system 50 of the present example comprises a field generator assembly 60, which comprises a set of magnetic field generators 64 that are integrated into a horseshoe-shaped frame 62. The field generators 64 are operable to generate alternating magnetic fields of different frequencies around the head (H) of the patient (P). An instrument may be inserted into the head (H) of the patient (P). Such an instrument may include one or more position sensors as described in greater detail below. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, the entirety of which is incorporated by reference herein. In some other variations, the patient (P) lies on a table, and the field generator assembly 60 is positioned on or near the table.

The IGS navigation system 50 of the present example further comprises a processor 52, which controls the field generators 64 and other elements of the IGS navigation system 50. For instance, the processor 52 is operable to drive the field generators 64 to generate alternating electromagnetic fields and process signals from the instrument to determine the location of a navigation sensor in the instrument within the head (H) of the patient (P). The processor 52 comprises a processing unit (e.g., a set of electronic circuits arranged to evaluate and execute software instructions using combinational logic circuitry or other similar circuitry) communicating with one or more memories. The processor 52 of the present example is mounted in a console 58, which comprises the operating controls 54 that include a keypad and/or a pointing device, such as a mouse or trackball. A physician uses the operating controls 54 to interact with the processor 52 while performing the surgical procedure.

While not shown, the instrument may include a navigation sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the navigation sensor of the instrument may comprise at least one coil at or near the distal end of the instrument. When such a coil is positioned within an alternating electromagnetic field generated by the field generators 64, the alternating magnetic field may induce an electrical current in the coil, and this induced electrical current may be communicated as a position-indicative signal along the electrical conduit(s) in the instrument and further to the processor 52 via coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position related signals (e.g., from induced currents) of the coil(s) in the instrument. Thus, a navigation sensor may serve as a position sensor by generating signals indicating the real-time position of the sensor within three-dimensional space or by otherwise indicating the real-time position of the sensor within three-dimensional space.

The processor 52 uses software stored in a memory of the processor 52 to calibrate and operate the IGS navigation system 50. Such operation includes driving the field generators 64, processing data from the instrument, processing data from the operating controls 54, and driving the display screen 56. In some implementations, operation may also include monitoring and enforcement of one or more safety features or functions of the IGS navigation system 50. The processor 52 is further operable to provide video in real time via display screen 56, showing the position of the distal end of the instrument in relation to a video camera image of the patient's head (H), a CT scan image of the patient's head (H), and/or a computer-generated three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, the display screen 56 may provide images in accordance with at least some of the teachings of U.S. Pat. No. 10,463,242, the entirety of which is incorporated by reference herein. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

### II. Example of Dilation Instrument With Translatable Features

In some scenarios, it may be desirable to advance a dilation catheter into an anatomical passageway in or near the ear, nose, or throat of a patient and expand the dilator to thereby dilate the passageway. For instance, it may be desirable to dilate a paranasal sinus ostium or other passageway associated with drainage of a paranasal sinus cavity, a Eustachian tube, a stenotic region in an airway of a patient, etc. It may also be desirable to incorporate a guide into such an instrument, to assist in guiding the dilation catheter into the targeted anatomical passageway, and to allow the dilation catheter to translate longitudinally relative to the guide. This may allow the guide to be initially positioned in relation to a targeted anatomical passageway while the dilation catheter is in a proximal position. The dilation catheter may then be advanced relative to the guide to a distal position to thereby enter the targeted anatomical passageway.

### A. Examples of Components of Dilation Instrument

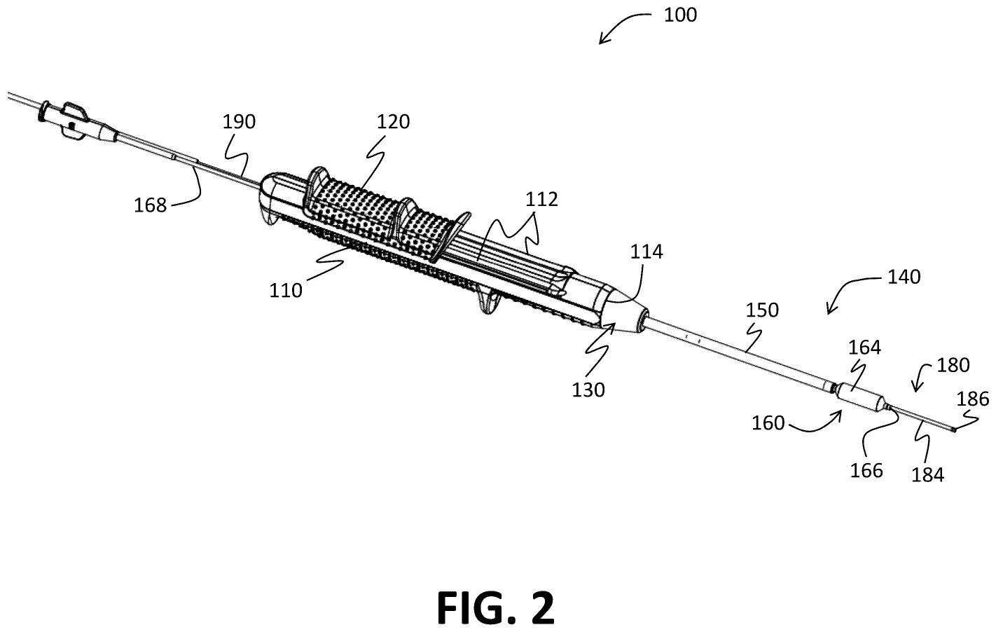

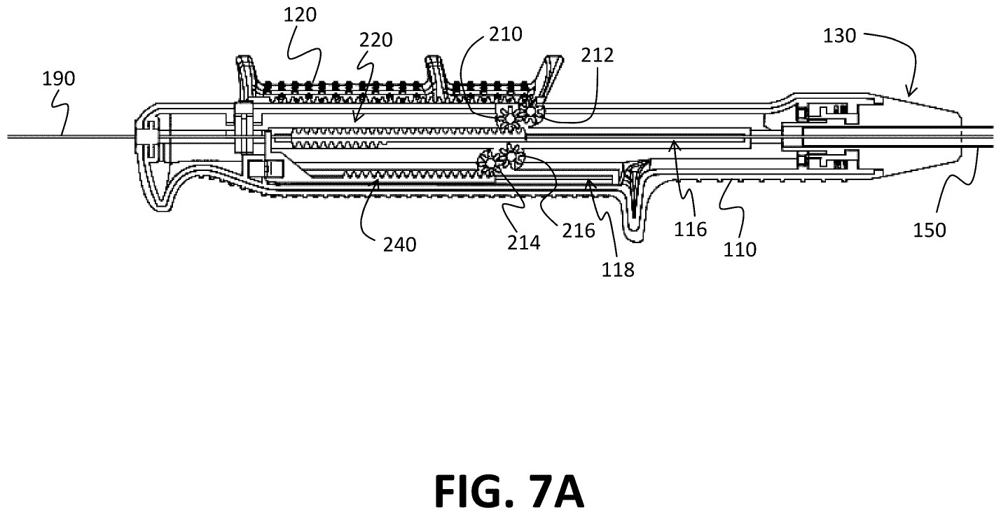

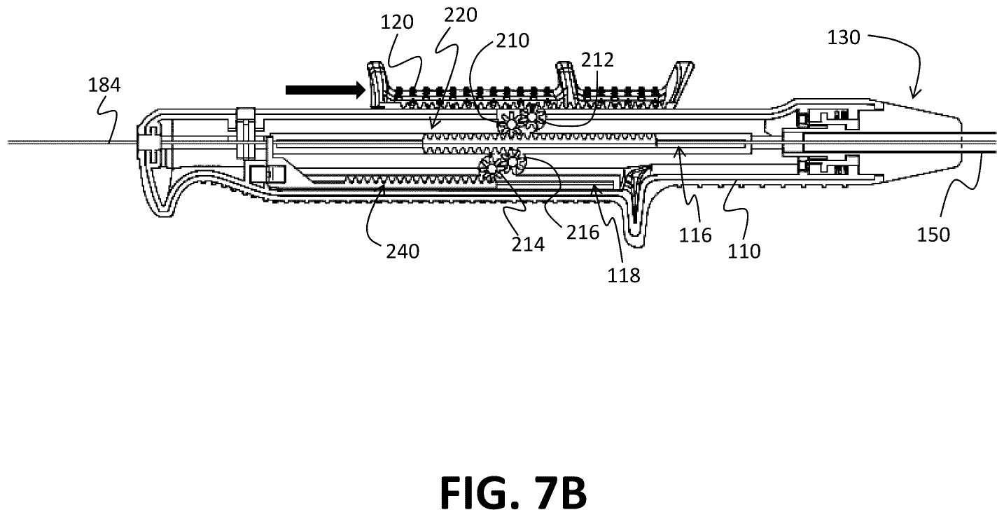

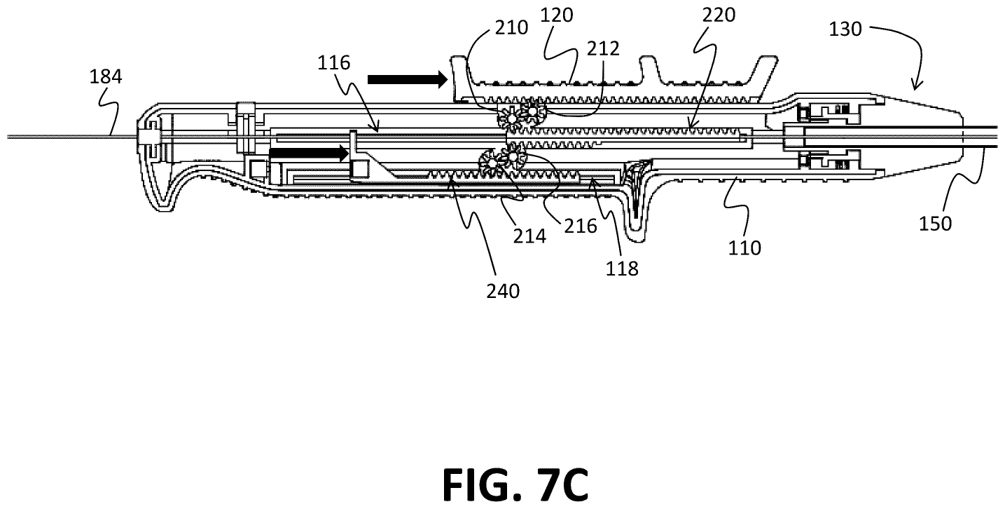

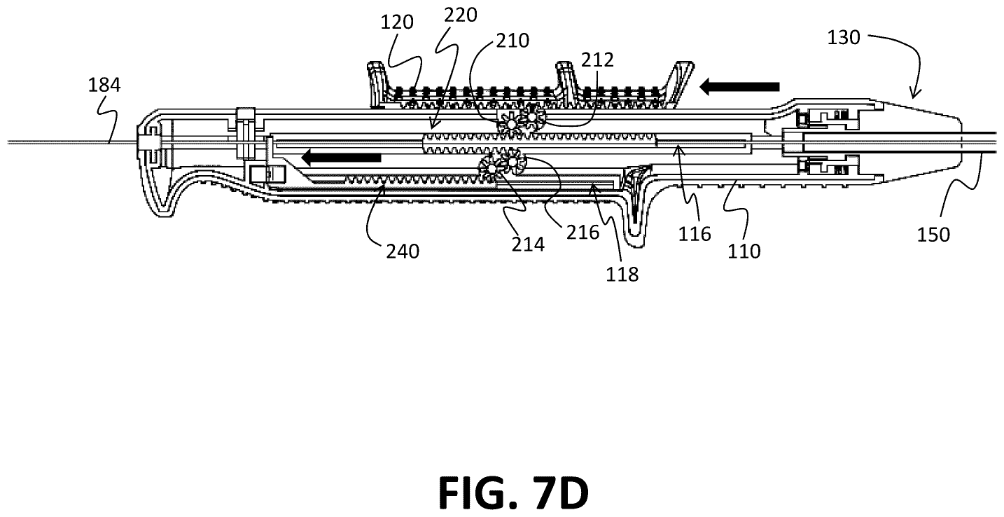

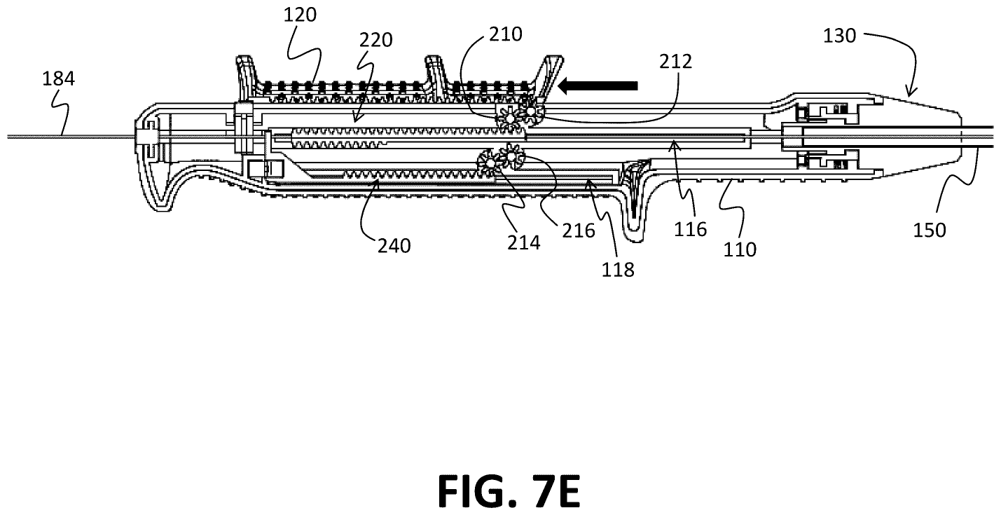

FIG. 2 shows a dilation instrument 100 that includes a body in the form of a handle 110, a guide rail actuation knob 130, and a shaft assembly 140. A dilation catheter actuator 120 is slidably supported on a pair of longitudinally extending rails 112 that protrude outwardly from the handle 110. The dilation catheter actuator 120 is configured to slide longitudinally along the rails 112 to drive longitudinal movement of a working element in the form of a dilation catheter assembly 160 relative to the handle 110, as will be described in greater detail below with reference to FIGS. 7A-7E.

The guide rail actuation knob 130 is positioned distally relative to the distal end 114 of the handle 110 in this example. The guide rail actuation knob 130 is movably coupled with the handle 110 such that the guide rail actuation knob 130 is operable to translate and rotate relative to the handle 110. Some components of the shaft assembly 140 are fixedly secured to the guide rail actuation knob 130, such that these components of the shaft assembly 140 will rotate and translate with the guide rail actuation knob 130 relative to the handle 110. In some other variations, the guide rail actuation knob 130 is only operable to translate relative to the handle 110. In still other variations, the guide rail actuation knob 130 is only operable to rotate relative to the handle 110. In still other variations, the knob 130 does not rotate or translate relative to the handle 110.

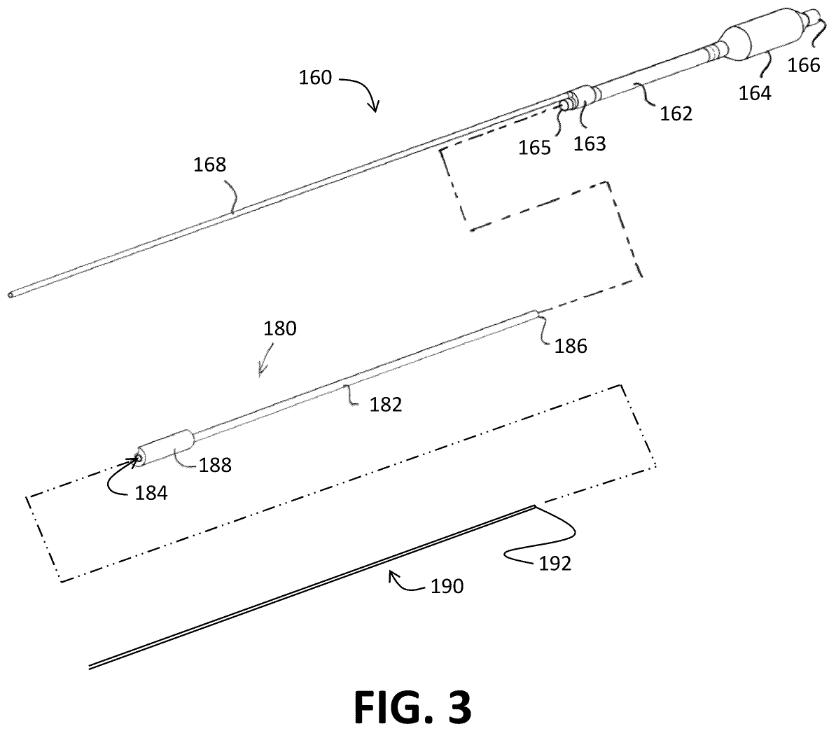

As shown in FIGS. 2-3, the shaft assembly 140 of the present example includes an outer sheath 150, a dilation catheter assembly 160, a guide rail assembly 180, and a guide element 190. The outer sheath 150 comprises a rigid tube that is fixedly secured to the guide rail actuation knob 130, such that the outer sheath 150 will translate and rotate unitarily with the guide rail actuation knob 130 in this example. Portions of the dilation catheter assembly 160 and the guide rail assembly 180 extend distally from the outer sheath 150. In the present example, the outer sheath 150 does not enter the head of the patient during operation of the instrument 100, though some scenarios may exist where the outer sheath 150 enters the patient during operation of the instrument 100.

The dilation catheter assembly 160 includes a shaft assembly 162, a balloon 164 proximal to a distal end 166 of the shaft assembly 162, a manifold 163, and a drive rod 168. The balloon 164 may comprise a non-extensible material and may be sized and configured to fit within a targeted anatomical passageway while in the deflated state; then dilate the targeted anatomical passageway while in the inflated state. The proximal end of the drive rod 168 is coupled with a source of inflation fluid (e.g., saline, etc.) and defines a lumen (not shown) that is in fluid communication with the balloon 164 via the manifold 163 and a fluid communication lumen (not shown) within the shaft assembly 162. Thus, inflation fluid may be communicated from the fluid source to the balloon 164 via the drive rod 168, the manifold 163, and the shaft assembly 162.

The guide rail assembly 180 includes a malleable guide rail 182 with an inner lumen 184, an atraumatic distal tip 186, and a proximal hub 188. In some versions, the distal tip 186 is dome shaped. In some other versions, the distal tip 186 is enlarged (e.g., configured as a ball tip or blueberry tip, etc.). The proximal end of the guide rail 182 is fixedly secured within the proximal hub 188, such that the guide rail 182 and the proximal hub 188 will move together unitarily. The guide rail assembly 180 is fixedly secured to the outer sheath 150 via the proximal hub 188, which is disposed within the interior of the outer sheath 150. The guide rail assembly 180 is thus fixedly secured relative to the guide rail actuation knob 130, such that the guide rail assembly 180 and the outer sheath 150 will rotate and translate with the guide rail actuation knob 130 relative to the handle 110. In the present example, no portion of the guide rail assembly 180 enters the handle 110 or is otherwise positioned proximally relative to the distal end 114 of the handle 110 at any stage of operation of the instrument 100. The guide rail 182 is slidably disposed within a lumen 165 of the shaft assembly 162 of the dilation catheter assembly 160, such that the dilation catheter assembly 160 is slidable along the guide rail 182 as described in greater detail below.

The guide rail 182 of the present example is malleable. The malleability of the guide rail 182 allows the guide rail 182 to be bent to a desired bend angle before being inserted into the head (H) of the patient (P). The malleability of the guide rail 182 may allow the guide rail 182 to maintain the bend angle while the guide rail 182 is disposed in the head of the patient, including while the dilation catheter assembly 160 is advanced distally relative to the guide rail 182. Such operability of the guide rail 182 may promote access by the dilation catheter assembly 160 to various locations within the head (H) of a patient (P), such as the maxillary sinus ostium, the frontal recess, the sphenoid sinus ostium, the Eustachian tube, etc., based on the selected bend angle. By way of example only, the bending of the guide rail 182 may be performed in accordance with at least some of the teachings of U.S. Pat. No. 11,013,897, the entirety of which is incorporated by reference herein.

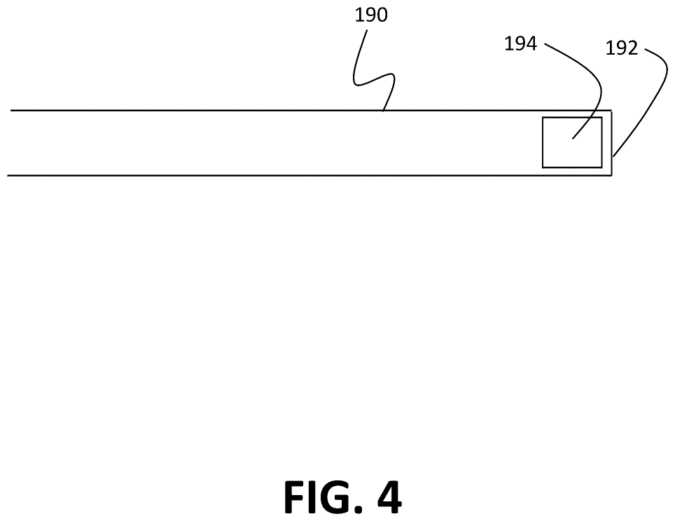

As best seen in FIGS. 3-4, the guide element 190 has a distal tip 192 with an indicator element 194 that is configured to indicate the position of the distal tip 192 in three-dimensional space. The guide element 190 may be in the form of a guidewire comprising metallic and/or polymeric material. The guide element 190 is slidably disposed within the lumen 184 of the guide rail 182, such that the guide element 190 is translatable within the guide rail 182 as described in greater detail below. In the present example, the indicator element 194 includes one or more position sensors that provide position-indicative signals to the IGS navigation system 50. Such position sensors may include one or more coils. When such a coil is positioned within an alternating electromagnetic field generated by the field generators 64, the alternating magnetic field may induce electrical current in the coil, and this induced electrical current may be communicated as a position-indicative signal via one or more wires, conductive traces, or other electrically conductive elements along the guide element 190. Signals from the indicator element 190 may be further communicated to the processor 52 via wire or wirelessly.

The processor 52 may determine the location of the distal tip 192 within a three-dimensional space (i.e., within the head (H) of the patient (P), etc.). To accomplish this, the processor 52 may execute an algorithm to calculate location coordinates of the distal tip 192 from the position related signals (e.g., from induced currents) of the coil(s) of the indicator element 194. Thus, a position sensor of the indicator element 194 may generate signals indicating the real-time position of the sensor within three-dimensional space or by otherwise indicating the real-time position of the sensor within three-dimensional space.

In addition to including a position sensor, or as an alternative to including a position sensor, the indicator element 194 may include an illuminating feature that is operable to project light outwardly to thereby provide transillumination through the skin of the patient (P) when the distal tip 192 is positioned inside a patient (P). In some such versions, the illuminating feature includes an optically transmissive window that is optically coupled with one or more optical fibers, with such one or more optical fibers being optically coupled with a light source. In some other versions, the illuminating feature includes one or more LEDs or other local sources of light positioned locally at the distal tip 192. It should be understood that the indicator element 194 may comprise a position sensor, an illuminating feature, or a combination of a position sensor and an illuminating feature.

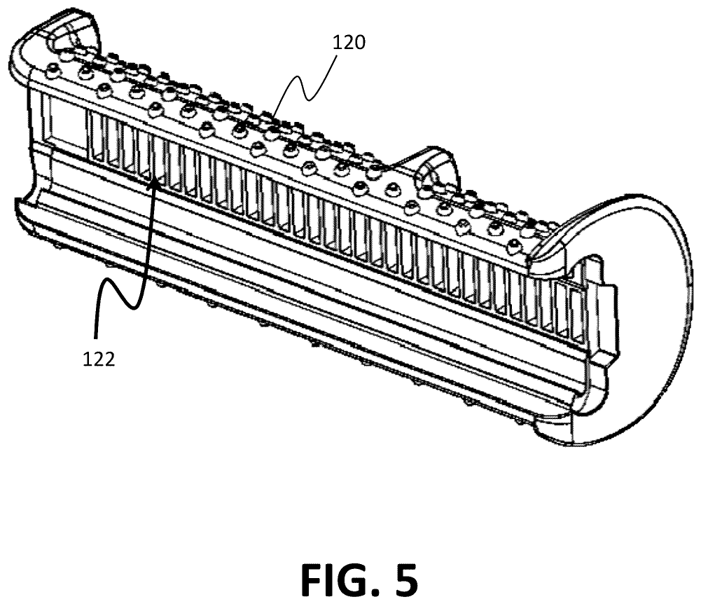

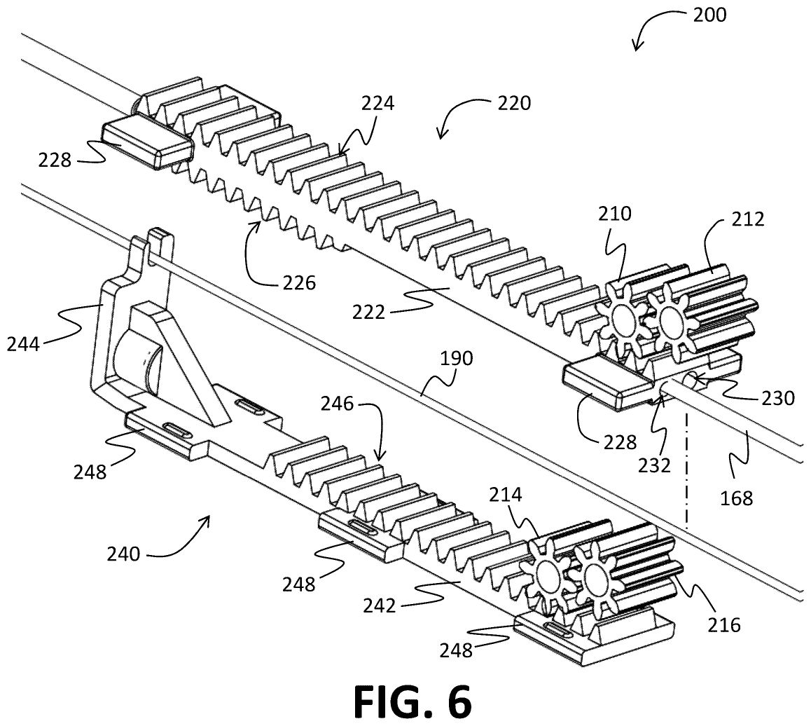

FIGS. 5-7E show components that may be used to actuate the dilation catheter assembly 160 and the guide element 190 relative to the handle 110. As shown in FIG. 5, the underside of the dilation catheter actuator 120 includes an integral rack 122. The teeth of the rack 122 are configured to mesh with the teeth of a pinion 212 that are exposed through an upper opening of the handle 210 as described in greater detail below. FIG. 6 shows the actuation assembly 200 components that are integrated into the handle 110, including a set of rack members 220, 240 and pinions 210, 212, 214, 216. The first rack member 220 includes an elongate body 222 having an integral upper rack 224 and an integral lower rack 226. A set of rail segments 228 extend outwardly from the body 222 and are configured to slidably fit in the upper channels 116 in the handle 110, such that the handle 110 slidably supports the first rack member 220. The body 222 further defines a first passageway 230 and a second passageway 232. In the present example, the drive rod 168 is fixedly secured within the second passageway 232, such that the dilation catheter assembly 160 translates unitarily with the first rack member 220 relative to the handle 110.

The guide element 190 is slidably disposed in the first passageway 230. The guide element 190 is not fixed in the first passageway 230, such that the guide element 190 may translate relative to the first rack member 220 during a portion of the range of motion when the first rack member 220 translates relative to the handle 110 as described in greater detail below. After exiting the distal end of the first passageway 230, the guide element 190 continues to extend distally into the proximal end of the guide rail 182. As noted above, the proximal end of the guide rail 182 is located in the guide rail hub assembly 130, at a longitudinal position that is distal to the distal end 114 of the handle 110.

The second rack member 240 includes an elongate body 242 having an integral upper rack 246 and a proximal upright portion 244. A set of rail segments 248 extend outwardly from the body 242 and are configured to slidably fit in the lower channels 118 of the handle 110, such that the handle 110 slidably supports the second rack member 240. The guide element 190 is fixedly secured to the proximal upright portion 244, such that the guide element 190 translates unitarily with the second rack member 240 relative to the handle 110.

The pinions 210, 212, 214, 216 are rotatably mounted to corresponding axles within the handle 110, such that each pinion 210, 212, 214, 216 is rotatable within the handle 110. As noted above, a portion of the pinion 212 protrudes through an upper opening of the handle 110 for meshing engagement with the rack 122 of the dilation catheter actuator 120. Thus, as the actuator 120 is translated along the handle 110 between the proximal position (FIG. 7A) and a distal position (FIG. 7C), this meshing engagement between the rack 122 and the pinion 212 causes the pinion 212 to rotate about its axle. The pinion 212 is also in meshing engagement with the pinion 210, such that rotation of the pinion 212 about its axle causes the pinion 210 to rotate about its own axle. The pinion 210 is also in meshing engagement with the upper rack 224 of the first rack member 220, such that rotation of the pinion 210 causes the first rack member 220 to translate longitudinally. Due to these meshing relationships between the racks 122, 224 and the pinions 210, 212, distal advancement of the actuator 120 along the handle 110 provides distal translation of the first rack member 220 within the handle 110, and proximal advancement of the actuator 120 along the handle 110 provides proximal translation of the first rack member 220 within the handle 110.

During a certain stage of operation of the actuation assembly 200 as described in greater detail below, the lower rack 226 of the first rack member 220 eventually achieves meshing engagement with the pinion 216, such that translation of the first rack member 220 drives rotation of the pinion 216 about its axle. The pinion 216 is also in meshing engagement with the pinion 214, such that rotation of the pinion 216 about its axle causes the pinion 214 to rotate about its own axle. The pinion 214 is also in meshing engagement with the rack 246 of the second rack member 240, such that rotation of the pinion 214 causes the second rack member 240 to translate longitudinally.

### B. Example of Operation of Dilation instrument

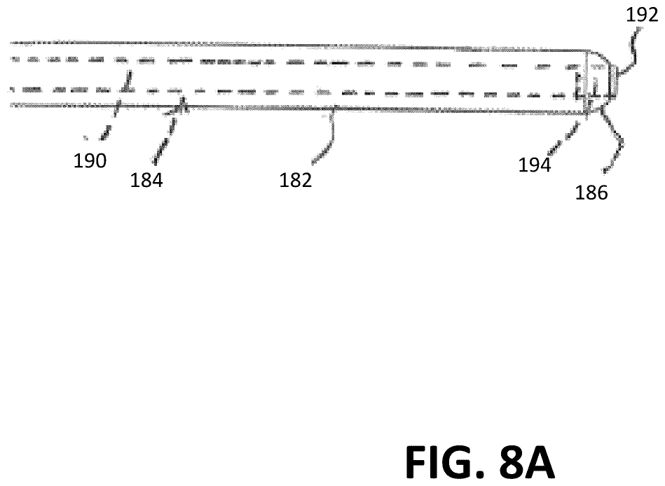

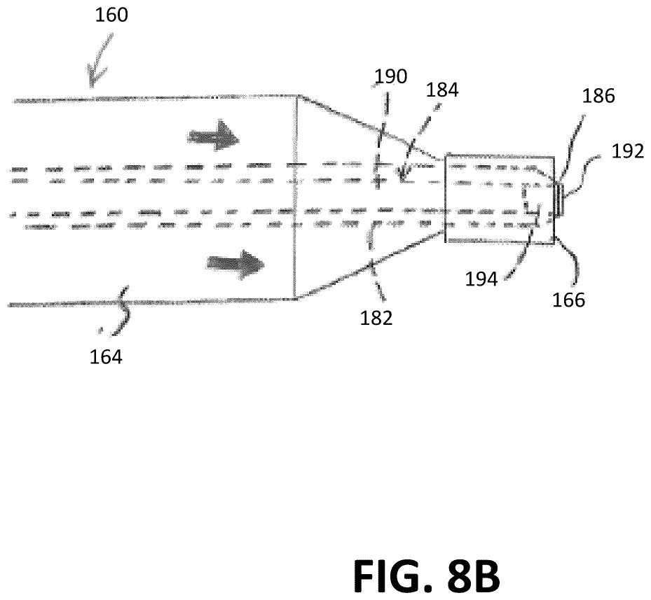

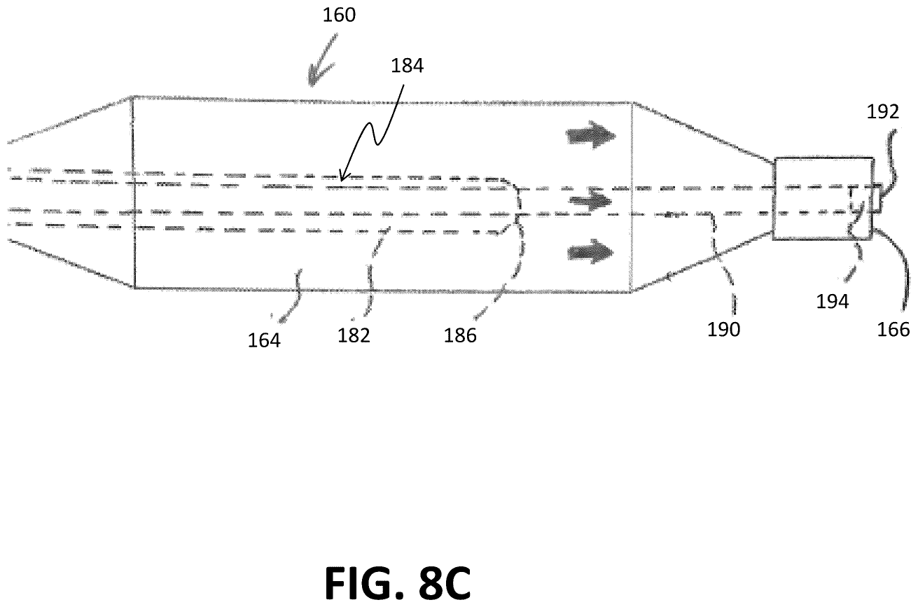

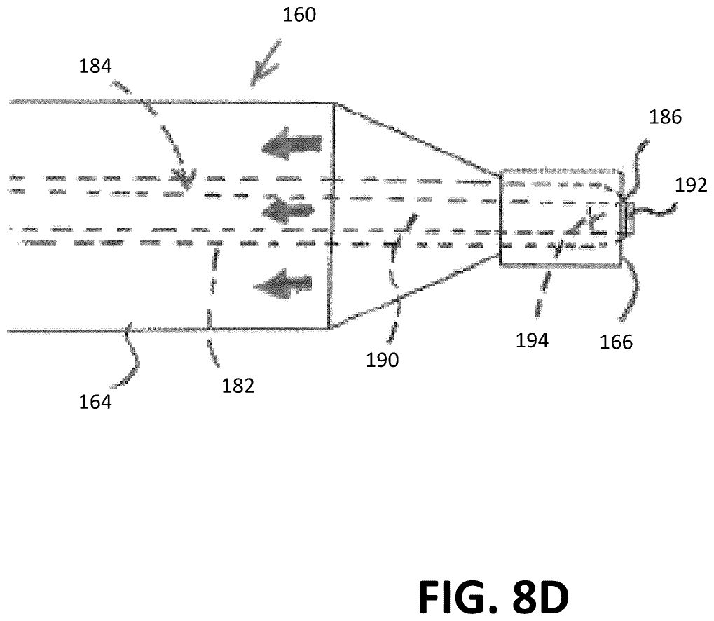

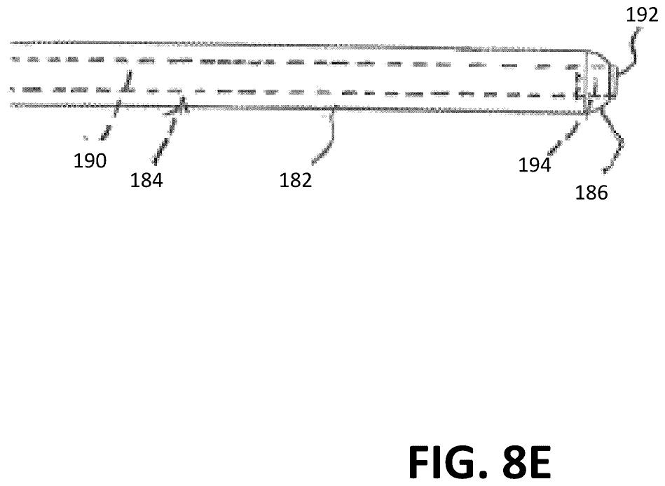

FIGS. 7A-7E show the actuation assembly 200 at various stages of operation based on the longitudinal position of the actuator 120 along the handle 110. FIGS. 8A-8E show components of the shaft assembly 140 at the same stages of operation. FIGS. 7A and 8A show the actuation assembly 200 and the shaft assembly 140 in an initial stage of operation, where the dilation catheter assembly 160 is in a proximal position. As shown in FIG, 7A, the actuator 120 and the first rack member 220 are proximally positioned along and within the handle 110 at this stage. As shown in FIG. 8A, the distal tip 192 of the guide element 190 is positioned at the distal tip 186 of the guide rail 182. Also in this state, the distal end 166 of the dilation catheter assembly 160 is spaced proximally from the distal tips 186, 192. Thus, a distal region of the guide rail 182 is exposed relative to the dilation catheter assembly 160. While the guide rail 182 is shown in a straight configuration in FIG. 8A, the guide rail 182 may alternatively be in a bent configuration as noted above. The operator may select and execute the desired bend angle in the guide rail 182 based on the location and/or configuration of the targeted anatomical passageway. The shaft assembly 140 may be inserted into the patient (P) while the dilation catheter assembly 160 is in the proximal position shown in FIG. 8A. With the distal tip 192 of the guide element 190 positioned at the distal tip 186 of the guide rail 182, the real-time position feedback provided by the indicator element 194 may effectively indicate the real-time position of the distal tip 186 in three-dimensional space.

Once the operator has sufficiently positioned the distal tip 186 at the desired location, based on feedback from the indicator element 194, the operator may begin advancing the actuator 120 distally along the handle 110 as shown in FIG. 7B. This distal advancement of the actuator 120 drives the first rack member 220 distally as shown in FIG. 7B. The distal advancement of the first rack member 220 drives the dilation catheter assembly 160 distally as shown in FIG. 8B. During the distal range of travel of the dilation catheter assembly 160, the distal end 166 eventually reaches the same longitudinal position of the distal tips 186, 192, as shown in FIG. 8B. Up to this point of operation, the second rack member 240 and the guide element 190 remain stationary, as the lower rack 226 has not yet started to drive rotation of the pinion 216, though the lower rack 226 has just made initial contact with the pinion 216.

As the operator continues to advance the actuator 120 distally along the handle 110 as shown in FIG. 7C, this further advancement of the actuator 120 drives the first rack member 220 further distally as shown in FIG. 7C. Moreover, since the lower rack 226 is now in meshing engagement with the pinion 216, the further advancement of the actuator 120 drives the second rack member 240 distally with the first rack member 220. Thus, as shown in FIG. 8C, as the dilation catheter assembly 160 continues to advance distally, the guide element 190 translates distally with the dilation catheter assembly 160, maintaining the distal tip 192 of the guide element 190 at the same longitudinal position as the distal end 166 of the dilation catheter assembly 160. Thus, with the distal tip 192 of the guide element 190 positioned at the distal end 166 of the dilation catheter assembly 160, the real-time position feedback provided by the indicator element 194 may effectively indicate the real-time position of the distal end 166 in three-dimensional space.

After reaching the distal position shown in FIGS. 7C and 8C, the operator may inflate the balloon 164 to dilate the targeted anatomical passageway. In some cases, the balloon 164 may be inflated and deflated repeatedly to achieve sufficient dilation. While the balloon 164 is shown in the inflated state in FIGS. 8B-8D, it should be understood that the balloon 164 may in fact be in a deflated state during the stages of operation shown in FIGS. 8B and 8D. Similarly, the balloon 164 may in fact be in a deflated state during part(s) of the stage of operation shown in FIG. 8C.

When the dilation is complete, the balloon 164 may be deflated and retracted proximally as shown in the transition from FIGS. 7C and 8C to FIGS. 7D and 8D. As shown, the dilation catheter assembly 160 eventually reaches the position where the distal end 166 reaches the same longitudinal position of the distal tips 186, 192. At this point, the lower rack 226 disengages the pinion 216 as shown in FIG. 7E, such that the guide element 190 ceases further proximal movement while the dilation catheter assembly 160 continues to translate proximally and such that the distal tip 192 of the guide element 190 remains at the distal tip 186 of the guide rail 182 as shown in FIG. 8E.

In view of the foregoing, during the stage of operation between that shown in FIGS. 7A and 8A and that shown in FIGS. 7B and 8B, the real-time position feedback provided by the indicator element 194 may effectively indicate the real-time position of the distal tip 186 in three-dimensional space. During the stage of operation between that shown in FIGS. 7B and 8B and that shown in FIGS. 7C and 8C, the real-time position feedback provided by the indicator element 194 may effectively indicate the real-time position of the distal end 166 in three-dimensional space. During the stage of operation between that shown in FIGS. 7C and 8C and that shown in FIGS. 7D and 8D, the real-time position feedback provided by the indicator element 194 may continue to effectively indicate the real-time position of the distal end 166 in three-dimensional space. During the stage of operation between that shown in FIGS. 7D and 8D and that shown in FIGS. 7E and 8E, the real-time position feedback provided by the indicator element 194 may again effectively indicate the real-time position of the distal tip 186 in three-dimensional space.

### III. Example of Features for Tracking Relative Movement Between Instrument Components

As described above, some instruments may include position sensors, such as coils that provide signals indicating a real-time position of the coil in three-dimensional space in response to an alternating electromagnetic field, thereby indicating a real-time position of a corresponding portion of the instrument in three-dimensional space. For instance, signals from a position sensor may indicate the real-time position of a first component of an instrument to/in which the position sensor is secured, as well as the real-time position of other portions of the instrument that are in a fixed, predetermined spatial relationship with the first component of the instrument. However, some instruments may include a second component that is movable relative to the first component and/or is otherwise not in a fixed, predetermined spatial relationship with the first component. In some such cases, an additional position sensor may be secured to the second component. This may increase cost, complexity, or other undesirable results.

As an alternative to adding a second position sensor for a second component of the instrument, the instrument may include a tracking structure or feature that is configured to track relative movement between the first component and the second component. To the extent that such a tracking feature is incapable of independently providing a signal that indicates a real-time position of the second component in three-dimensional space, a processor may nevertheless process signals from the position sensor of the first component and a signal from the tracking feature to thereby determine the real-time position of the second component. Examples of how a tracking feature may be incorporated into an instrument are described in greater detail below.

### A. First Example of Tracking Feature to Account for Relative Movement Between Instrument Components

As described above, the dilation instrument 100 includes a guide element 190 with an indicator element 194 that may include one or more position sensors that allow the processor 52 to determine the real-time position of the distal tip 192 within three-dimensional space. As also described above, the same position-indicative signals associated with the indicator element 194 may also effectively indicate the real-time position of the distal tip 186 of the guide rail assembly 180 (e.g., during the stages of operation shown in FIGS. 8A-8B and 8D-8E) or the real-time position of the distal end 166 of the dilation catheter assembly 160 (e.g., during the stages of operation shown in FIGS. 8B-8D). There may be instances where there are deviations in the relative position between the guide element 190 and the dilation catheter assembly 160.

For example, when the dilation catheter assembly 160 is at a proximal position or in an intermediate longitudinal position (e.g., before the lower rack 226 engages the pinion 216), it may be possible for an operator to intentionally (or inadvertently) move the guide element 190 longitudinally relative to the dilation catheter assembly 160. Further, there may be situations where the operator may engage a portion of the guide element 190 protruding proximally from the handle 110 and thereby urge the guide element 190 distally.

In accordance with at least some embodiments disclosed herein is the realization that if such relative movement between the guide element 190 and the dilation catheter assembly 160 occurs outside the sequence of operation described above with reference to FIGS. 7A-7E and 8A-8E, the position-indicative signals associated with the indicator element 194 may no longer effectively indicate the real-time position of the distal end 166 of the dilation catheter assembly 160 (e.g., during the stages of operation shown in FIGS. 8B-8D). In some cases, the processor 52 is intended to drive the display screen 56 to show the real-time position of the distal end 166 in relation to a video camera image of the patient's head (H), a CT scan image of the patient's head (H), and/or a computer-generated three-dimensional model of the anatomy within and adjacent to the patient's nasal cavity during the stages of operation shown in FIGS. 8B-8D, based on position-indicative signals associated with the indicator element 194. The ability to accurately provide such a display may be adversely affected if the position-indicative signals associated with the indicator element 194, alone, no longer effectively indicate the real-time position of the distal end 166 of the dilation catheter assembly 160 (e.g., during the stages of operation shown in FIGS. 8B-8D). It may therefore be desirable to separately track movement of the dilation catheter assembly 160, such that the dilation catheter assembly 160 movement tracking data may be factored in with the position-indicative signals associated with the indicator element 194 to thereby determine the real-time position of the distal end 166 of the dilation catheter assembly 160. Such movement tracking data may thus be used to account for any unintentional or otherwise out-of-sequence relative movement between the guide element 190 and the dilation catheter assembly 160.

Due to significant restrictions in size and complexity, conventional systems have not been able to separately track movement of one component of an instrument relative to another component without increasing the size, cost, and complexity of the device. Thus, systems and components within the scope of the embodiments disclosed herein provide a significant advancement and benefit due to the unique configurations and designs that have been created and described in the present disclosure. For example, as an alternative to adding a second position sensor to the dilation catheter assembly 160 to track the movement of the dilation catheter assembly 160 relative to other parts of the instrument 100, the instrument 100 can have a tracking feature that provides a signal indicating the movement of the dilation catheter assembly 160 relative to other parts of the instrument 100 (e.g., the guide element 190). As described in greater detail below, the components that make up the tracking feature are unobtrusive and can be integrated into various parts of the body the instrument 100 so as to minimize the intrusiveness of the instrument 100 to the patient. Further, the tracking feature evades the need for additional sensors in the instrument 100 which limits the cost and the complexity associated with incorporating expensive and fragile electronics into the instrument 100.

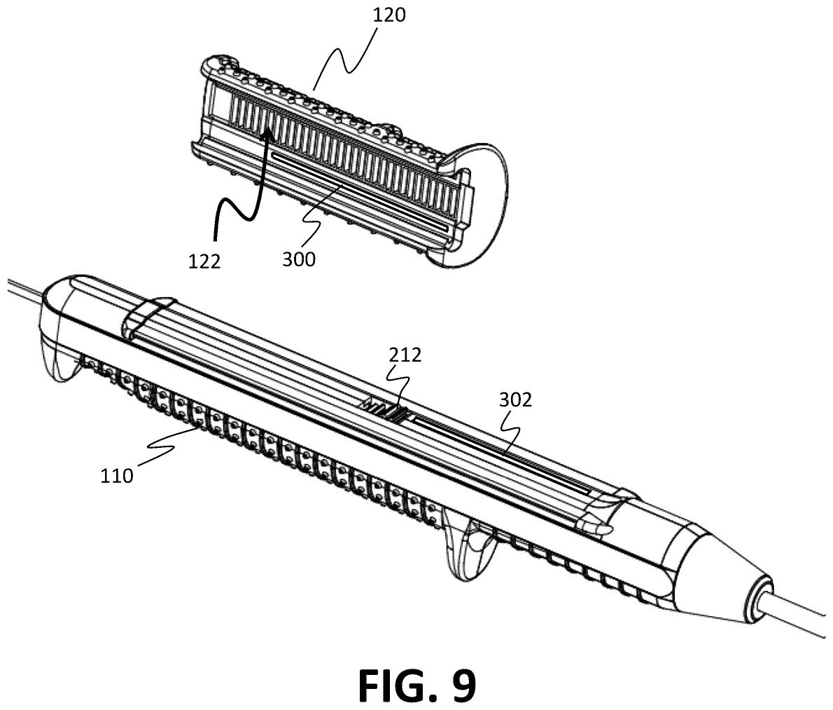

FIG. 9 shows one example of how a tracking feature may be integrated into the instrument 100 to track movement of the dilation catheter assembly 160. In this example, the tracking feature includes a first capacitor plate 300 and a second capacitor plate 302. The first capacitor plate 300 is mounted to the underside of the actuator 120. The second capacitor plate 302 is mounted to an upper surface of the handle 110. As shown in FIG. 9, the first and second capacitor plates 300, 302 have a small profile that minimizes interference with other components of the instrument 100. The thin, generally cylindrical shape of the first and second capacitor plates 300, 302 facilitates different embodiments of the tracking feature with the first and second capacitor plates 300, 302 being incorporated in other parts of the instrument 100. A few of these different embodiments are described in greater detail below.

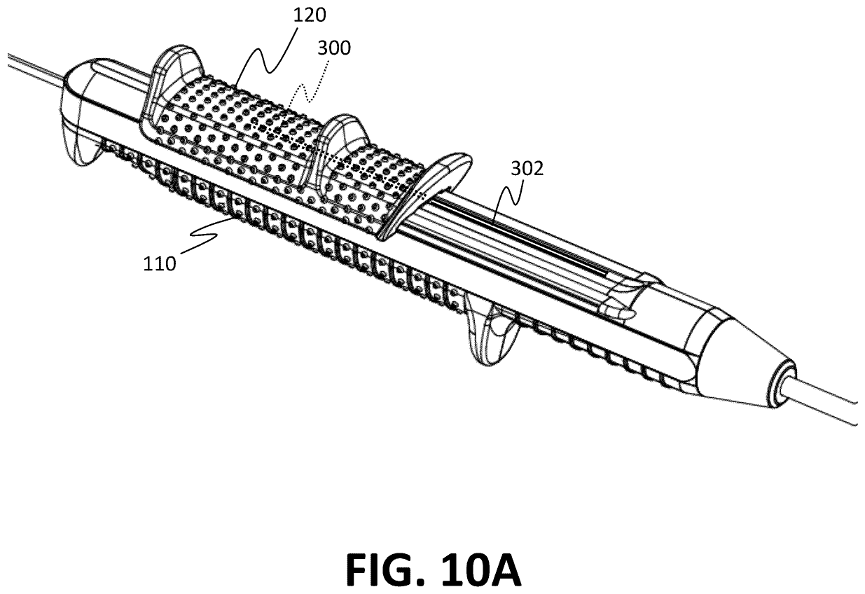

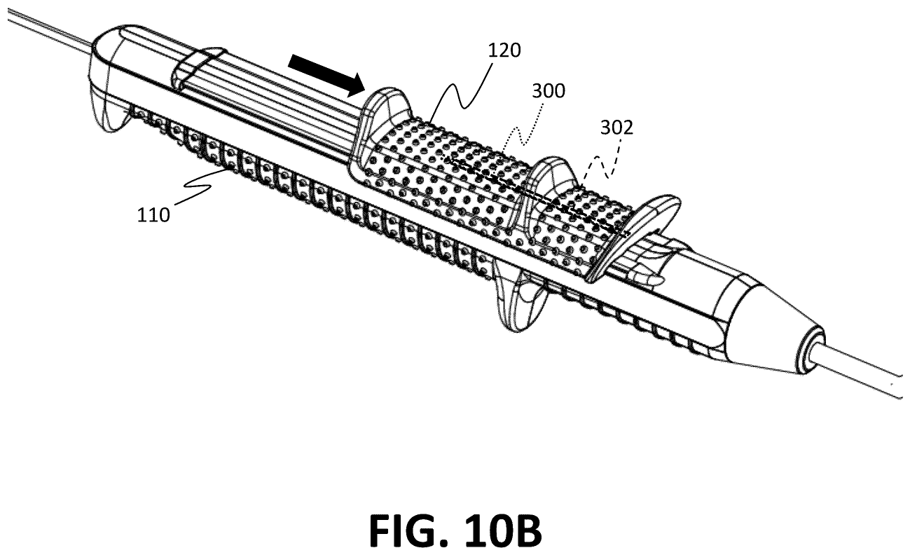

The capacitor plates 300, 302 are positioned to overlap, with the degree of overlap varying based on the longitudinal position of the actuator 120 along the handle 110. For instance, in the operational state shown in FIG. 10A, the actuator 120 is in a proximal-most position, and the capacitor plate 300 is not disposed over the capacitor plate 302. At this operational state, when a voltage is applied to the capacitor plates 300, 302, the capacitor plates 300, 302 do not provide a capacitance. In some other variations, there is some degree of overlap between the capacitor plates 300, 302 when the actuator 120 is in the proximal-most position of FIG. 10A, and this overlap provides a baseline capacitance associated with the actuator 120 being in the proximal-most position.

As the operator advances the actuator 120 distally along the handle 110, the degree of overlap between the capacitor plates 300, 302 changes progressively. In the example shown in FIG. 10B, the overlap between the capacitor plates 300, 302 is maximized when the actuator 120 is in a distal-most position. Thus, when a voltage is applied to the capacitor plates 300, 302 at this stage of operation, the capacitance provided by the capacitor plates 300, 302 may be maximized. During operation, a voltage may be continuously applied to the capacitor plates 300, 302, such that the capacitance provided by the capacitor plates 300, 302 may progressively change as actuator translates between the proximal-most position (FIG. 10A) and the distal-most position (FIG. 10B).

The processor 52 may track the capacitance provided by the capacitor plates 300, 302 to thereby track the position of the dilation catheter assembly 160 relative to the handle 110. The processor 52 may also track the position-indicative signals associated with the indicator element 194 as described above. By obtaining a first signal—i.e., an initial position reading—at the start of operation (e.g., when the guide element 190 is in the proximal-most position shown in FIG. 8A), the processor 52 may effectively determine the position of the handle 110 in three-dimensional space. Utilizing this information, the processor 52 may effectively determine the real-time position of the distal end 166 of the dilation catheter assembly 160 based on a second signal—i.e., the real-time capacitance signal—from the tracking feature provided by the capacitor plates 300, 302 in combination with the real-time signals associated with the indicator element 194. The processor 52 may thus account for any relative movement between the guide element 190 and the dilation catheter assembly 160 that is inadvertent or otherwise out-of-sequence. Conventional devices have not been able to account for such relative movement because incorporating additional sensors into a device increases the size of the device, which makes the device more intrusive to patients. Thus, the tracking feature and the capacitor plates 300, 302 disclosed herein facilitate real-time movement tracking of the dilation catheter assembly 160 relative to the handle 110 and/or the guide element 190 without adding an additional sensor to the dilation catheter assembly 160, which would compromise the safety and comfort of the patient.

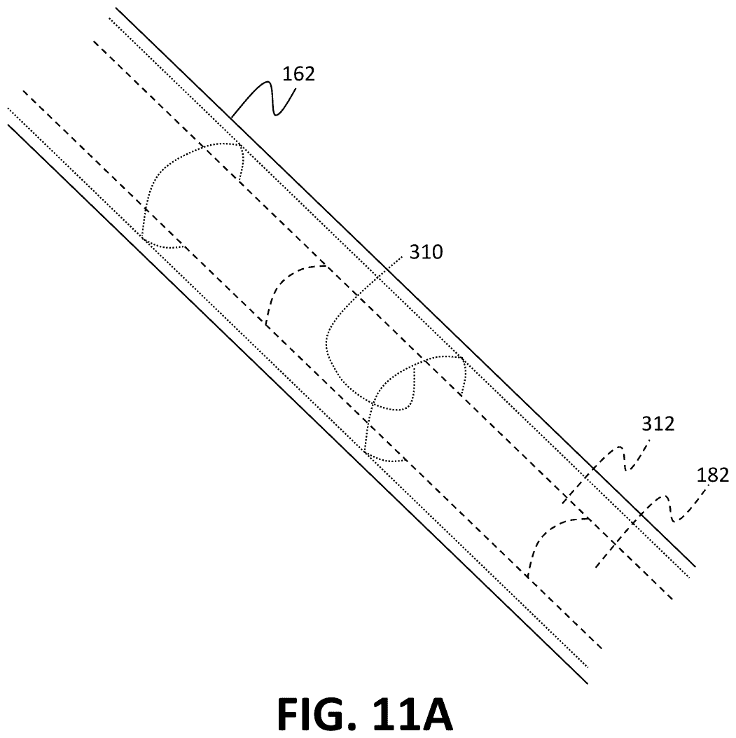

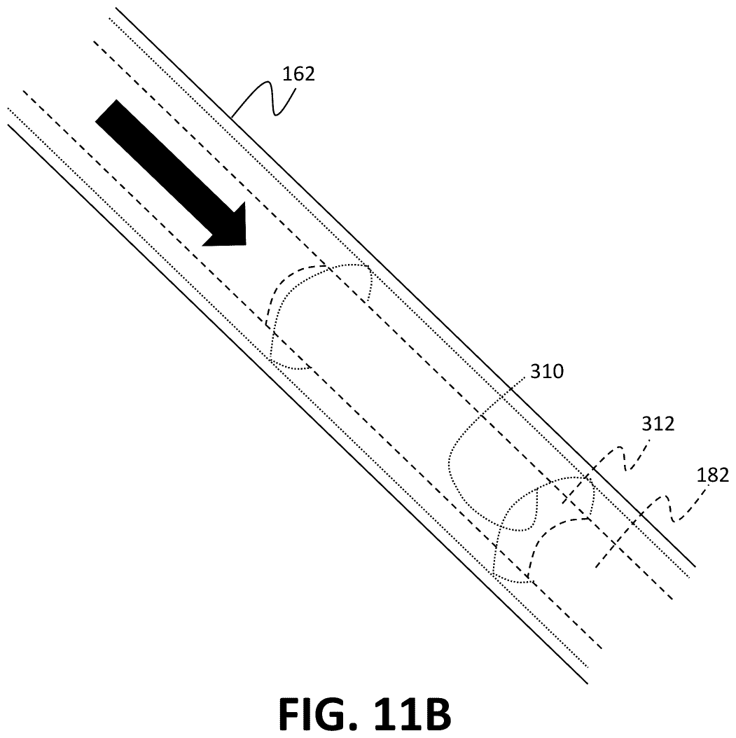

While the capacitor plates 300, 302 are integrated into the actuator 120 and the handle 110 in this example, the capacitor plates 300, 302 have a profile that allows them to be integrated elsewhere within instrument. For instance, one capacitor plate may be fixed within the interior of the handle 110 while another capacitor plate may be fixed to the first rack member 220. As another example, capacitor plates may be integrated within the shaft assembly 140. An example of such an implementation is shown in FIGS. 11A-11B. As shown, a first capacitor plate 310 is secured within an inner diameter of the shaft assembly 162 of the dilation catheter assembly 160 while another capacitor plate 312 is secured to an outer diameter of the guide rail 182. The capacitor plates 310, 312 of this example are generally cylindrical. FIG. 11A shows a state of operation where the dilation catheter assembly 160 is in a proximal position while FIG. 11B shows a state of operation where the dilation catheter assembly 160 is in a distal position. As shown, the degree of overlap between the capacitor plates 310, 312 progressively increases as the dilation catheter assembly 160 is advanced distally, such that the capacitance defined by the plates 310, 312 may indicate the real-time longitudinal position of the dilation catheter assembly 160 relative to the guide rail 182.

To the extent that the guide rail 182 remains stationary relative to the handle 110 as the dilation catheter assembly 160 is translated longitudinally along the guide rail 182, the capacitance defined by the plates 310, 312 may also indicate the real-time longitudinal position of the dilation catheter assembly 160 relative to the handle 110. The processor 52 may thus track the capacitance provided by the capacitor plates 310, 312 to thereby track the position of the dilation catheter assembly 160 relative to the handle 110. In accordance with the description provided above with respect to the tracking feature provided by the capacitor plates 300, 302, the processor 52 may effectively determine the real-time position of the distal end 166 of the dilation catheter assembly 160 based on the real-time capacitance signal from the tracking feature provided by the capacitor plates 310, 312 in combination with the real-time signals associated with the indicator element 194. The processor 52 may thus account for any relative movement between the guide element 190 and the dilation catheter assembly 160 that is inadvertent or otherwise out-of-sequence. This may enable the processor 52 to more accurately drive the display screen 56 to show the real-time position of the distal end 166 in relation to a video camera image of the patient's head (H), a CT scan image of the patient's head (H), and/or a computer-generated three-dimensional model of the anatomy within and adjacent to the patient's nasal cavity during the stages of operation shown in FIGS. 8B-8D, particularly when the guide element 190 has encountered any unintentional or otherwise out-of-sequence movement relative to the dilation catheter assembly 160.

It should be understood that some variations of the dilation instrument 100 may utilize the capacitor plates 300, 302 to track movement of the dilation catheter assembly 160 relative to the handle 110 in addition to utilizing the capacitor plates 310, 312 to track movement of the dilation catheter assembly 160 relative to the guide rail 182. Alternatively, the dilation instrument 100 may only include capacitor plates in connection with the handle 110 or only in connection with the guide rail 182. In addition to, or as an alternative to, utilizing capacitor plates 300, 302, 310, 312 to track movement of the dilation catheter assembly 160 relative to the handle 110 and/or relative to the guide rail 182, capacitor plates may be used to track movement of the guide element 190 relative to the handle 110 and/or relative to the guide rail 182. For instance, one capacitor plate may be fixed within the interior of the handle 110 while another capacitor plate may be fixed to the second rack member 240. As another example, one capacitor plate may be fixed to an outer diameter of the guide element 190 while another capacitor plate is fixed to an inner diameter of the inner lumen 184 of the guide rail 182.

As yet another example of a variation, capacitor plates may be used to track movement of the guide element 190 relative to the dilation catheter assembly 160. For instance, one capacitor plate may be fixed to the first rack member 220 while another capacitor plate may be fixed to the second rack member 240. As another example, one capacitor plate may be fixed to an outer diameter of the guide element 190 while another capacitor plate is fixed to an inner diameter of the dilation catheter assembly 160. In some such variations, the capacitor plates may be positioned near the distal end 166 and the distal tip 192, and the capacitor plates may only provide a capacitance during stages of operation where the distal end 166 and the distal tip 192 are both distal to the distal tip 186. Such capacitor plates may still indicate any longitudinal offsets between the guide element 190 relative to the dilation catheter assembly 160 that may occur from movement of the guide element 190 relative to the dilation catheter assembly 160 that occurs inadvertently or otherwise out-of-sequence. Moreover, the processor 52 may still effectively determine the real-time position of the distal end 166 of the dilation catheter assembly 160 based on the real-time capacitance signal from the tracking feature provided by such capacitor plates in combination with the real-time signals associated with the indicator element 194.

While the foregoing examples provide a tracking feature that indicates movement between components based on changes in capacitance, it should be understood that tracking features may have other configurations and may indicate movement based on other principles. For instance, a tracking feature may provide an electrical resistance value that varies based on the relative position between components that move relative to each other, such that the real-time relative position may be determined based on the real-time electrical resistance value. In some such versions, the tracking feature includes a linear potentiometer. As another example, a tracking feature may include an optical sensor, such as an optical linear encoder or optical rotary encoder, etc., where the optical signal changes based on changes in the relative position between components that move relative to each other. As another example, one or more proximity sensors may be used to track the real-time relative position between components that move relative to each other. Alternatively, a tracking feature may take any other suitable form. In any of these cases, the signal from the alternative tracking feature may be used as described above with reference to the capacitance signals provided by plates 300, 302, 310, 312.

### B. Example of Capacitive Tracking for Supplementing Position Sensor Data

While the above example describes how a tracking feature may be used to account for relative movement between components that is either inadvertent or otherwise outside of a standard operational sequence of movement, a similar tracking feature may also be used to account for intentional movement that is within a standard operational sequence of movement, to thereby determine the real-time position of a second component (which lacks a position sensor) based on the real-time position of a first component (which includes a position sensor).

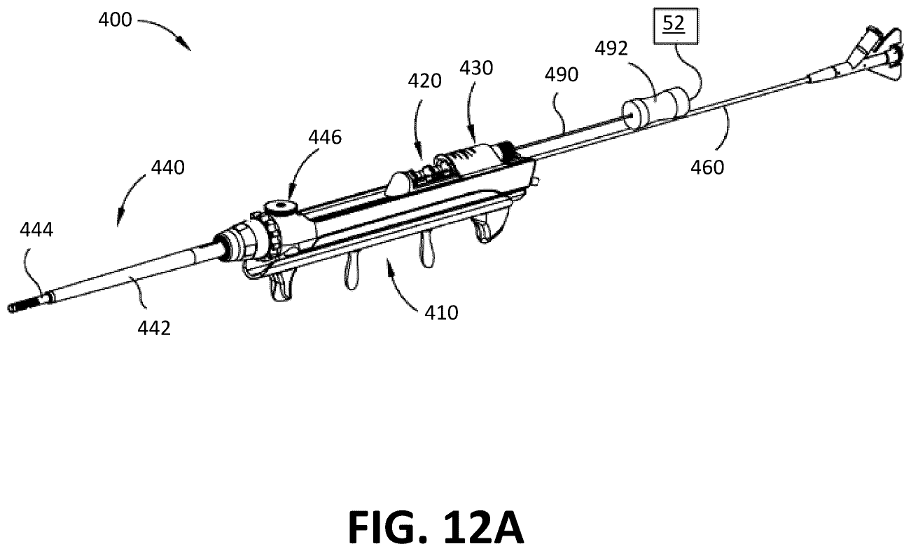

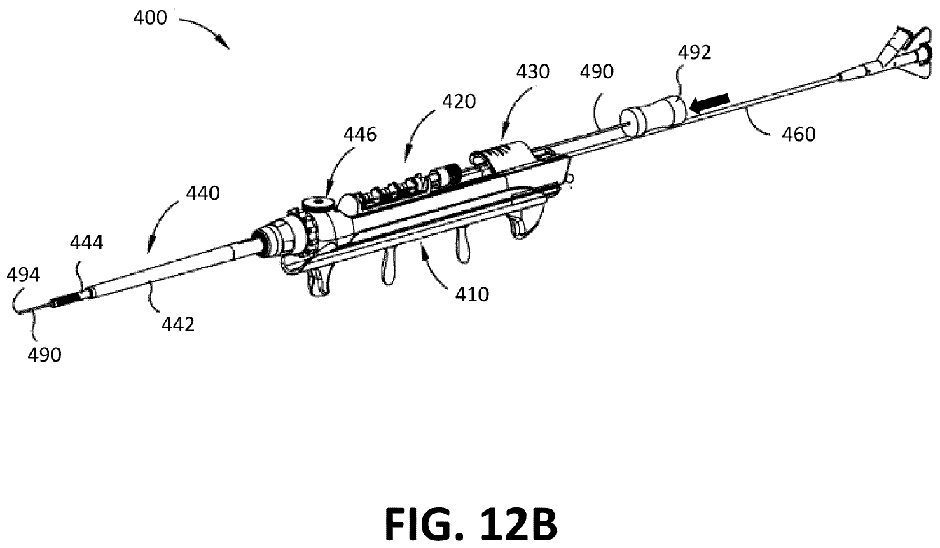

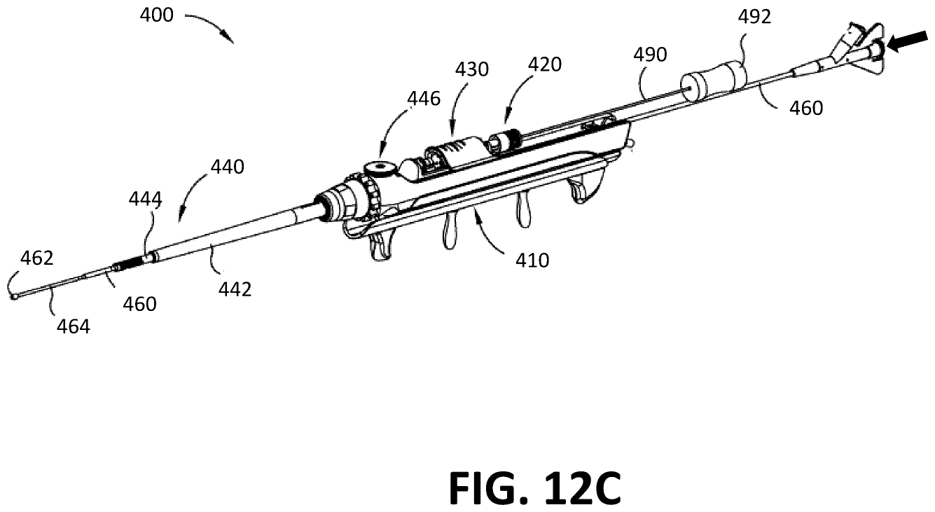

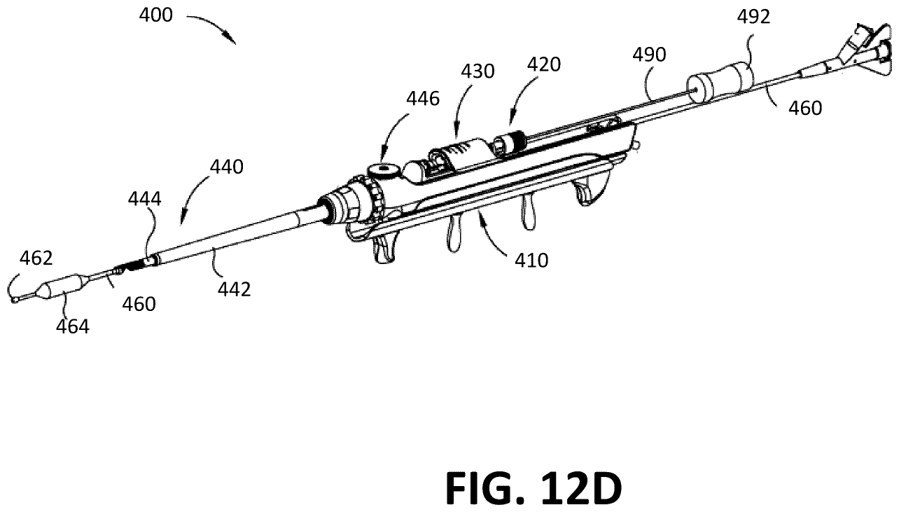

FIGS. 12A-12D show an example of a dilation instrument 400 that has independently actuated components where only one of the independently actuated components has an integral position sensor. The dilation instrument 400 of this example includes a body in the form of a handle 410, a first actuator 420, a second actuator 430, and a shaft assembly 440. Shaft assembly 440 includes a rigid proximal portion 442 and a steerable distal portion 444. A third actuator 446 on the handle 410 is operable to drive deflection of the steerable portion 444 (e.g., via pull-wire). A guide element 490 is coupled with the first actuator 420, such that the first actuator 420 may be translated relative to the handle 410 between a proximal position (FIG. 12A) and a distal position (FIGS. 12B-12D) to thereby drive the guide element 490 between a proximal position and a distal position. A working element in the form of a dilation catheter assembly 460 is coupled with the second actuator 430, such that the second actuator 430 may be translated relative to the handle 410 between a proximal position (FIGS. 12A-12B) and a distal position (FIGS. 12C-12D) to thereby drive the dilation catheter assembly 460 between a proximal position and a distal position.

The guide element 490 may be configured similar to the guide element 190, such that the guide element 490 may be in the form of a guidewire comprising metallic and/or polymeric material and may include an indicator element similar to the indicator element 194 at or near a distal tip 494 of the guide element 490. Such an indicator element may include one or more position sensors (e.g., one or more coils) that provide position-indicative signals to the IGS navigation system 50, as described above. Such position sensors may include one or more coils. As shown in FIG. 12A, the guide element 490 may be coupled with the processor 52 via a connector 492, which may provide a wired coupling or a wireless coupling.

The dilation catheter assembly 460 may be configured similar to the dilation catheter assembly 160, such that the dilation catheter assembly 460 includes a distal tip 462 and a balloon 464 just proximal to the distal tip 462.

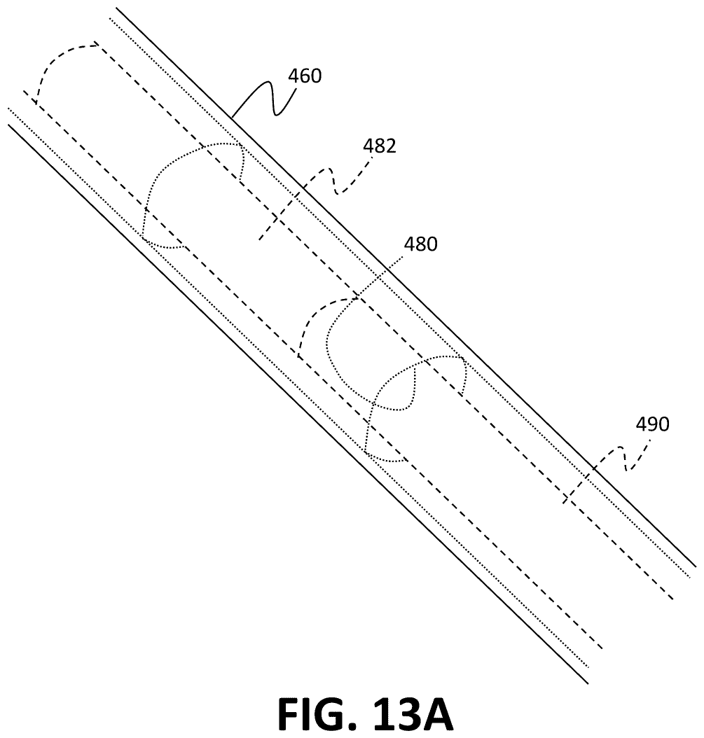

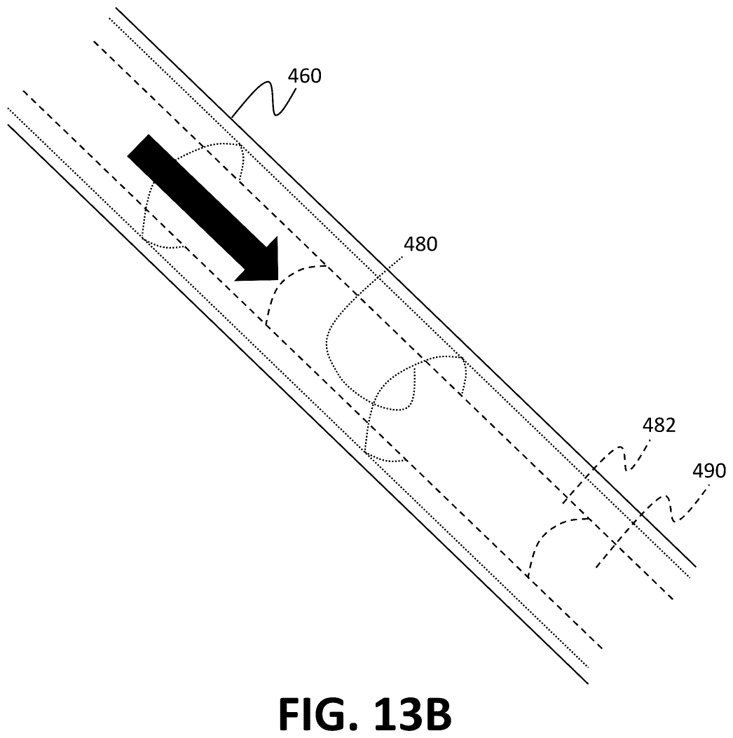

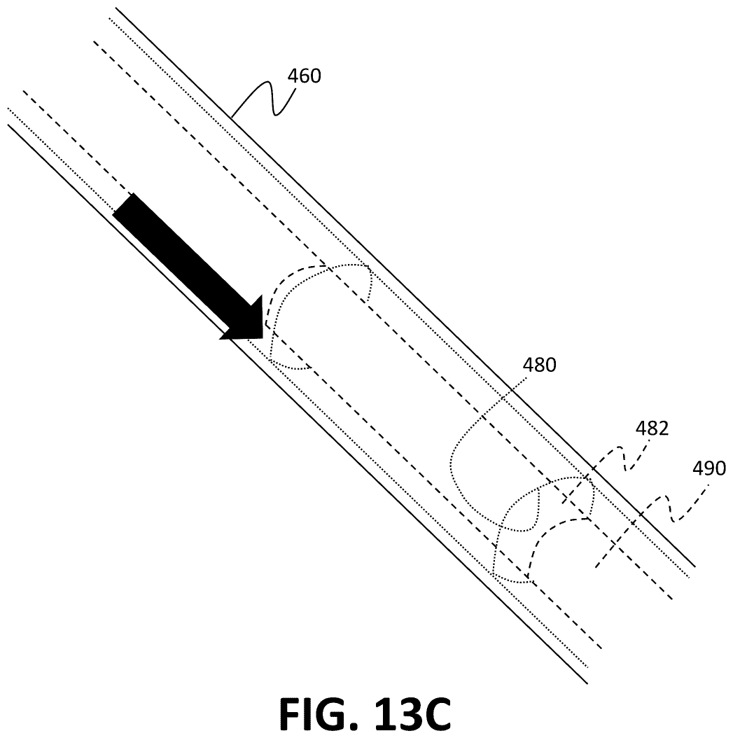

It should be understood from the foregoing that the dilation instrument 400 may be configured and operable similar to the dilation instrument 100, except that the dilation catheter assembly 460 and the guide element 490 are actuatable independently via respective actuators 430, 420 rather than being actuated in a certain sequence by a single the actuator 120. Since the guide element 490 is independently actuated by its own actuator 420 in this example, and since the guide element 490 has an integral position sensor while the dilation catheter assembly 460 does not, it may be particularly beneficial for the dilation instrument 400 to include a tracking feature that provides tracking of relative movement between the guide element 490 and the dilation catheter assembly 460. To that end, FIGS. 13A-13C show a tracking feature in the form of the capacitor plates 480, 482. The capacitor plate 480 is fixed to an inner diameter in the dilation catheter assembly 460 while the capacitor plate 482 is fixed to an outer diameter of the guide element 490.

FIG. 13A shows an operational state similar to that shown in FIG. 12A, where the guide element 490 is in a proximal position and where the dilation catheter assembly 460 is in a proximal position. As shown, the capacitor plates 480, 482 provide a certain degree of overlap in this operational state, resulting in a corresponding capacitance that is indicative of the guide element 490 and the dilation catheter assembly 460 both being in respective proximal positions. FIG. 13B shows an operational state similar to that shown in FIG. 12B, where the guide element 490 is in a distal position while the dilation catheter assembly 460 remains in the proximal position. As shown, the capacitor plates 480, 482 provide another certain degree of overlap in this operational state, resulting in a corresponding capacitance that is indicative of the guide element 490 being in a distal position while the dilation catheter assembly 460 remains in the proximal position. FIG. 13C shows an operational state similar to that shown in FIGS. 12C-12D, where the guide element 490 is in the distal position and where the dilation catheter assembly 460 is in a position. As shown, the capacitor plates 480, 482 provide another certain degree of overlap in this operational state, resulting in a corresponding capacitance that is indicative of the guide element 490 and the dilation catheter assembly 460 both being in respective distal positions.

It should be understood that the capacitance defined by the capacitor plates 480, 482 progressively changes as the guide element 490 translates between the proximal position and the distal position and as the dilation catheter assembly 460 translates between the proximal position and the distal position. The processor 52 may thus track the capacitance provided by the capacitor plates 480, 482 to thereby track the position of the dilation catheter assembly 460 relative to the handle 410. In accordance with the description provided above with respect to the tracking feature provided by the capacitor plates 300, 302, 310, 312, the processor 52 may effectively determine the real-time position of the distal end 462 of the dilation catheter assembly 460 based on the real-time capacitance signal from the tracking feature provided by the capacitor plates 480, 482 in combination with the real-time signals associated with the position sensor that is at or near the distal tip 494 of the guide element 490. In some cases, this may enable the processor 52 to drive the display screen 56 to simultaneously show the real-time position of the distal tip 494 and the distal end 462 in relation to a video camera image of the patient's head (H), a CT scan image of the patient's head (H), and/or a computer-generated three-dimensional model of the anatomy within and adjacent to the patient's nasal cavity. In addition, or in the alternative, the data from the position sensor of the guide element 460 and/or the data from the tracking feature provided by the capacitor plates 480, 482 may be utilized in any other suitable fashion.

While the tracking feature of the dilation instrument 400 is shown and described as being positioned on/in the dilation catheter assembly 460 and the guide element 460, some variations may provide tracking features elsewhere, in addition to (or as an alternative to) providing a tracking feature on/in the dilation catheter assembly 460 and the guide element 460. For instance, one capacitive plate may be fixed to the first actuator 420 while another capacitor plate is fixed to the handle 410, where the degree of overlap between these capacitor plates (and the resulting capacitance) varies based on the longitudinal position of the first actuator 420 along the handle 410. In addition, or in the alternative, one capacitive plate may be fixed to the second actuator 430 while another capacitor plate is fixed to the handle 410, where the degree of overlap between these capacitor plates (and the resulting capacitance) varies based on the longitudinal position of the first actuator 430 along the handle 410.

As noted above in the context of the capacitor plates 300, 302, 310, 312, the capacitor plates 480, 482 may be substituted or supplemented with any other suitable kind(s) of tracking feature(s). For instance, the dilation instrument 400 may include a tracking feature that utilizes electrical resistance values, optical tracking, proximity sensing, and/or any other suitable form(s) of tracking feature(s).

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: An apparatus, comprising: (a) a body; (b) a shaft assembly extending distally from the body; (c) a guide element having a position sensor, the guide element being translatable relative to the body, the position sensor being configured to provide a signal indicating a position of the guide element in three-dimensional space; (d) a working element, the working element being translatable relative to the body and relative to the guide element; and (e) a movement tracking feature, the movement tracking feature being configured to generate a signal indicating movement of the working element relative to one or both of the body or the guide element.

Example 2: The apparatus of Example 1, the movement tracking feature including: (i) a first component fixed relative to one or both of the body or the guide element, and (ii) a second component fixed relative to the working element.

Example 3: The apparatus of Example 2, further comprising an actuator, the actuator being movable relative to the body to drive longitudinal movement of the working element relative to the body.

Example 4: The apparatus of Example 3, the second component being fixed to the actuator.

Example 5: The apparatus of Example 2, further comprising a guide rail extending distally relative to the body, the working element being translatable relative to the guide rail.

Example 6: The apparatus of Example 5, the first component being fixed to the guide rail.

Example 7: The apparatus of Example 6, the second component being fixed to the working element.

Example 8: The apparatus of any of Examples 2 through 3 or 5, the first component being fixed to a distal portion of the guide element, the second component being fixed to a distal portion of the working element.

Example 9: The apparatus of any of Examples 2 through 8, the second component being positioned to overlap the first component with a degree of overlap varying based on a position of the working element relative to the body.

Example 10: The apparatus of any of Examples 2 through 9, the first component comprising a first capacitor plate, the second component comprising a second capacitor plate.

Example 11: The apparatus of Example 10, the movement tracking feature being configured to provide varying capacitance based on a position of the working element relative to the body.

Example 12: The apparatus of any of Examples 1 through 11, the body comprising a handle.

Example 13: The apparatus of any of Examples 1 through 12, the shaft assembly including an outer sheath.

Example 14: The apparatus of Example 13, the guide element and the working element extending through the shaft assembly to form part of the shaft assembly.

Example 15: The apparatus of any of Examples 13 through 14, the outer sheath including a rigid proximal portion and a steerable distal portion.

Example 16: The apparatus of any of Examples 1 through 4 or 6 through 15, the shaft assembly including a guide rail, the working element being translatable relative to the guide rail.

Example 17: The apparatus of Example 16, the guide rail having a malleable distal portion.

Example 18: The apparatus of any of Examples 16 through 17, the working element being positioned exteriorly about the guide rail.

Example 19: The apparatus of any of Examples 16 through 18, the guide element being positioned interiorly within the guide rail, the guide element being translatable relative to the guide rail.

Example 20: The apparatus of any of Examples 1 through 19, the guide element comprising a guidewire.

Example 21: The apparatus of any of Examples 1 through 20, the position sensor comprising a coil, the coil being configured to provide an induced current signal in response to an electromagnetic field.

Example 22: The apparatus of any of Examples 1 through 21, the working element comprising a dilation catheter assembly.

Example 23: The apparatus of Example 22, the dilation catheter assembly comprising a balloon.

Example 24: The apparatus of Example 23, the balloon being operable to dilate an anatomical passageway within a head of a patient.

Example 25: The apparatus of any of Examples 1 through 24, the guide element having a distal end, the position sensor being positioned at or near the distal end.

Example 26: The apparatus of any of Examples 1 through 25, further comprising a processor, the processor being configured to: (i) receive a signal from the position sensor of the guide element, (ii) receive a signal from the movement tracking feature, (iii) determine a real-time position of the guide element in three-dimensional space based on the signal from the position sensor of the guide element, and (iv) determine a real-time position of the working element in three-dimensional space based on a combination of the signal from the position sensor of the guide element and the signal from the movement tracking feature.

Example 27: The apparatus of Example 26, the processor being further configured to drive a display to simultaneously visually indicate the real-time position of the guide element in relation to an image associated with a patient and the real-time position of the working element in relation to the image.

Example 28: The apparatus of any or Examples 26 through 27, the processor being configured to: (i) determine a real-time position of a distal tip of the guide element in three-dimensional space based on the signal from the position sensor of the guide element, and (ii) determine a real-time position of a distal end of the working element in three-dimensional space based on a combination of the signal from the position sensor of the guide element and the signal from the movement tracking feature.

Example 29: The apparatus of any of Examples 1 through 28, further comprising an actuator, the actuator being movable through a first range of motion and through a second range of motion, the actuator being operable to drive translation of the working element relative to the body while the guide element remains stationary relative to the body as the actuator is moved through the first range of motion, the actuator being operable to drive simultaneous translation of the working element and the guide element relative to the body as the actuator is moved through the first range of motion.

Example 30: The apparatus of any of Examples 1 through 28, further comprising: (a) a first actuator, the first actuator being movable relative to the body to drive longitudinal movement of the working element relative to the body; and (b) a second actuator, the second actuator being movable relative to the body to drive longitudinal movement of the guide element relative to the body, the second actuator being operable independently relative to the first actuator.

Example 31: An apparatus, comprising: (a) a handle; (b) a shaft assembly extending distally from the handle; (c) a guide element having a position sensor, the guide element being translatable relative to the body, the position sensor being configured to provide a signal indicating a position of the guide element in three-dimensional space; (d) a dilation catheter assembly; and (e) a movement tracking feature, the movement tracking feature being configured to generate a signal indicating movement of the working element relative to one or both of the body or the guide element.

Example 32: The apparatus of Example 31, the movement tracking feature including: (i) a first component configured to move with one or both of the body or the guide element, and (ii) a second component configured to move with the dilation catheter assembly.

Example 33: The apparatus of Example 32, the first component being fixed to the body.

Example 34: The apparatus of Example 32, the first component being fixed to the guide element.

Example 35: The apparatus of any of Examples 32 through 34, the second component being fixed to the dilation catheter assembly.

Example 36: An apparatus, comprising: (a) a body; (b) a shaft assembly extending distally from the body; (c) a guide element having a position sensor, the guide element being translatable relative to the body; (d) a working element, the working element being translatable relative to the body and relative to the guide element; (e) a movement tracking feature, the movement tracking including: (i) a first component configured to move with one or both of the body or the guide element, and (ii) a second component configured to move with the working element, the movement tracking feature being further configured to provide a signal that varies based on a position of the working element relative to one or both of the body or the guide element; and (f) a processor, the processor being configured to: (i) receive a signal from the position sensor of the guide element, (ii) determine a real-time position of the guide element in three-dimensional space based on the signal from the position sensor of the guide element, (iii) receive a signal from the movement tracking feature, (iv) determine a real-time position of the working element in three-dimensional space based on a combination of the signal from the position sensor of the guide element and the signal from the movement tracking feature.

Example 37: A method, comprising: (a) inserting a shaft assembly into a patient; (b) advancing a guide element into the patient via shaft assembly, the shaft assembly extending distally from a body, the guide element having a position sensor, the position sensor providing a signal indicating a real-time position of the advanced guide element in three-dimensional space; and (c) advancing a working element into the patient via shaft assembly, a movement tracking feature providing a signal indicating movement of the working element relative to one or both of the body or the guide element during at least part of the advancing.

Example 38: The method of Example 37, the act of inserting a shaft assembly into a patient comprising inserting the shaft assembly into a head of the patient.

Example 39: The method of Example 38, the working element comprising a dilation catheter, the method further comprising dilating an anatomical passageway in the head of the patient with the dilation catheter.

Example 40: The method of any of Examples 37 through 39, the act of advancing the working element into the patient comprising providing relative movement between a first component of the movement tracking feature and a second component of the movement tracking feature, the first component being fixed relative to the body or the guide element, the second component moving with the working element.

Example 41: The method of Example 40, the first component comprising a first capacitor plate, the second component comprising a second capacitor plate, the relative movement between the first component of the movement tracking feature and the second component providing a change in capacitance defined by the first capacitor plate and the second capacitor plate.

Example 42: A method, comprising: (a) receiving a signal from a position sensor of a guide element while the guide element is inserted in a patient, the guide element being part of a medical instrument; (b) determining a real-time position of the guide element in three-dimensional space within the patient, based on the signal from the position sensor of the guide element; (c) receiving a signal from a movement tracking feature, the movement tracking feature being part of the medical instrument, the signal from the movement tracking feature indicating movement of a working element of the medical instrument relative to one or both of a body of the medical instrument or the guide element; and (d) determining a real-time position of the working element in three-dimensional space based on a combination of the signal from the position sensor of the guide element and the signal from the movement tracking feature.

Example 43: The method of Example 42, further comprising driving a display to simultaneously visually indicate the real-time position of the guide element in relation to an image associated with a patient and the real-time position of the working element in relation to the image.

Example 44: The method of any of Examples 42 through 43, the position sensor comprising a coil, the method further comprising providing an electromagnetic field around a portion of the patient, the signal from the position sensor of the guide element being induced by the electromagnetic field.

Example 45: The method of any of Examples 42 through 44, the signal from the movement tracking feature having a capacitance value, the capacitance value indicating movement of a working element of the medical instrument relative to one or both of a body of the medical instrument or the guide element.

### V. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present inventions, further adaptations of the methods and systems described herein may be accomplished by appropriate modifications by one skilled in the art without departing from the scope of the present inventions. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. A medical instrument, comprising:
a body comprising a lumen;
a shaft assembly extending distally from the body;
a guidewire extending through the lumen and having a position sensor configured to provide a first signal indicating a position of the guidewire in three-dimensional space;
a working element slidably coupled to the body and translatable in a distal direction along the guidewire; and
a movement tracker comprising a first component coupled to the working element and a second component coupled to at least one of the body or the guidewire, the tracker being configured to generate a second signal when the first component moves relative to the second component.

2. The medical instrument of claim 1, wherein the second component is coupled to the body, and the second signal indicates a movement of the working element relative to the body.

3. The medical instrument of claim 1, wherein the second component is coupled to the guidewire, and the second signal indicates a movement of the working element relative to the guidewire.

4. The medical instrument of claim 1, wherein the second component is coupled to the body and the guidewire, and the second signal indicates a movement of the working element relative to the body and the guidewire.

5. The medical instrument of claim 1, wherein the working element comprises an actuator that is movable relative to the body to drive longitudinal movement of the working element relative to the body, the first component is coupled to the actuator, and the second component is coupled to the body.

6. The medical instrument of claim 1, wherein the shaft assembly comprises a guide rail, the working element is translatable relative to the guide rail, and the second component is coupled to the guide rail.

7. The medical instrument of claim 6, wherein the guide rail comprises a malleable distal portion.

8. The medical instrument of claim 1, wherein the second component is coupled to a distal portion of the guidewire, and the first component is coupled to a distal portion of the working element.

9. The medical instrument of claim 1, wherein the first component overlaps the second component with a degree of overlap that varies based on a position of the working element relative to at least one of the body or the guidewire.

10. The medical instrument of claim 9, wherein the second signal is directly related to the degree of overlap.

11. The medical instrument of claim 1, wherein the first component comprises a first capacitor plate, the second component comprises a second capacitor plate, and the second signal is a capacitance.

12. The medical instrument of claim 11, wherein the tracker is configured to provide varying capacitance based on a position of the working element relative to the body to thereby track the position of the working element relative to the body.

13. The medical instrument of claim 1, wherein the working element comprises a dilation catheter assembly, and the dilation catheter assembly comprises a balloon that is operable to dilate an anatomical passageway within a head of a patient.

14. The medical instrument of claim 1, further comprising a processor configured to:
receive the first signal from the position sensor of the guidewire;
receive the second signal from the tracker;
determine a real-time position of the guidewire in three-dimensional space based on the first signal; and
determine a real-time position of the working element in three-dimensional space based on a combination of the first signal and the second signal.

15. The medical instrument of claim 1, further comprising an actuator that is movable through a first range of motion and a second range of motion, wherein the actuator is configured to:
drive translation of the working element relative to the body and the guidewire as the actuator is moved through the first range of motion; and
drive simultaneous translation of the working element and the guidewire relative to the body as the actuator is moved through the second range of motion.

16. The medical instrument of claim 1, further comprising:
a first actuator that is movable relative to the body to drive longitudinal movement of the working element relative to the body; and
a second actuator that is movable relative to the body to drive longitudinal movement of the guidewire relative to the body, the second actuator being operable independently relative to the first actuator.

17. A medical instrument, comprising:
a body comprising a lumen;
a guidewire extending through the lumen and having a position sensor;
a working element that movably coupled to the body and translatable along the guidewire; and
a movement tracker comprising a first component configured to move with the working element and a second component configured to move with at least one of the body or the guidewire,
wherein the first component overlaps the second component with a degree of overlap that varies based on the movement of the working element relative to at least one of the body or the guidewire,
wherein the tracker is configured to provide a signal, the signal being directly related to the degree of overlap.

18. A method for using a medical instrument with real-time position sensing, the method comprising:
inserting a shaft assembly of the medical instrument into a patient, wherein the shaft assembly extends distally from a body of the medical instrument;
advancing a guidewire through the body and the shaft assembly, wherein the guidewire has a position sensor configured to provide a signal indicating a real-time position of the guidewire in three-dimensional space;
translating a working element distally along the guidewire; and
in response to translating the working element, causing a movement tracker to provide a signal indicating movement of the working element relative to at least one of the body or the guidewire.

19. The method of claim 18, wherein translating the working element comprises moving a first component of the movement tracker relative to a second component of the movement tracker, the first component being coupled to the working element, and the second component being coupled to at least one of the body or the guidewire.

20. The method of claim 19, wherein the first component comprises a first capacitor plate, the second component comprises a second capacitor plate, and moving the first component relative to the second component comprises changing a capacitance between the first capacitor and the second capacitor.
